(19) United States
(12) Patent Application Publication
(10) Pub. No.: US 2013/0079305 A1
(43) Pub. Date: Mar. 28, 2013

(54) 3-SUBSTITUTED VINYLBORONATES AND USES THEREOF

(71) Applicants: Morris Srebnik, Mevasseret Zion (IL); Abed Al-Aziz Quntar, Jerusalem (IL); Alina Botvinik, Jerusalem (IL); Arie Dagan, Jerusalem (IL)

(72) Inventors: Morris Srebnik, Mevasseret Zion (IL); Abed Al-Aziz Quntar, Jerusalem (IL); Alina Botvinik, Jerusalem (IL); Arie Dagan, Jerusalem (IL)

(73) Assignee: Yissum Research Development Company of the Hebrew University of Jerusalem Ltd., Jerusalem (IL)

(21) Appl. No.: 13/700,766

(22) PCT Filed: May 31, 2011
(86) PCT No.: PCT/IL2011/000423

§ 371 Date: Nov. 29, 2012

Related U.S. Application Data
(60) Provisional application No. 61/349,924, filed on May 31, 2010.

(51) Int. Cl. A61K 31/69

(52) U.S. Cl. CPC A61K 31/69
USPC 514/64; 435/375; 558/288

(57) ABSTRACT

3-substituted vinylboronates and their use in the treatment of cancer such as colorectal cancer are disclosed. In some embodiments, the 3-substituted vinylboronates have the general Formula I:

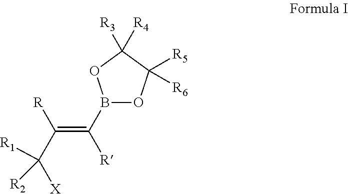

with the variables in the formula being as defined in the specification.

### FIELD AND BACKGROUND OF THE INVENTION

The present invention, in some embodiments thereof, relates to therapy and more particularly, but not exclusively, to a novel methodology for treating cancer such as colorectal cancer, which utilize boron-containing compounds.

Colorectal cancer is a malignant tumor that forms in the tissues of the colon or the rectum or both. Worldwide, colorectal cancer is the fourth most commonly diagnosed malignant disease, with an estimated 1,023,000 new cases and 529,000 deaths each year. Existing therapies for colorectal cancer are surgery, chemotherapy, radiation therapy and biological therapy. The choice of the treatment depends on the anatomical location of the tumor, on the stage of the cancer and the patient health condition. Commonly there are combinations between therapies. The search for new cancer drugs continues in order to discover compounds with higher cure levels and less adverse effects.

There are several colorectal cancer screening tests: standard or sensitive guaiac fecal occult-blood test, fecal immunochemical test, stool DNA, CT colonography, sigmoidoscopy and colonoscopy. The main prognostic indicator in colorectal cancer is the pathological stage of the cancer at the time it is discovered. The tumor-node-metastasis (TMN) system of the American Joint Committee on Cancer is now the most commonly used colorectal cancer staging system. The system is based on the staging of tumor invasion, nodal status and presence or absence of distant metastases.

The transition from normal epithelium to adenoma (a benign tumor of glandular origin) and to carcinoma (a malignant tumor arising from the epithelia) is associated with a progressive accumulation of major genetic alterations. There are major pathways that lead to colorectal cancer development. One pathway is chromosomal instability and the “suppressor pathway”, which accounts for about 85% of cases, and the second pathway, which accounts for about 15% of cases, is characterized by microsatellite instability. Cancers that develop through the first pathway develop from pre-existing adenomas after the acquisition of changes in a set of tumor-suppressor genes. Cancers that develop through the second pathway have defective DNA mismatch repair. This defect is related to a germline mutation or to failure to express a mismatch-repair gene. Tumors that grow through different routes differ one from another in appearance and behavior. The type of genetic origin of colorectal cancer may also influence its response to therapy.

The chemotherapeutic and biotherapeutic drugs that exist for colorectal cancer therapy are as follows.

5-Fluorouracil is a nucleoside analog that blocks thymidylate synthase, and thereby stops the synthesis of thymine nucleotides and DNA replication. 5-Fluorouracil is usually administered with leucovorin, a reduced folate, that stabilizes the binding of 5-fluorouracil to thymidylate synthase and as a result enhances the inhibition of DNA synthesis.

Capecitabine is an oral fluoropyrimidine prodrug. Thymidine phosphorylase converts capecitabine to 5-fluorouracil at the site of the tumor.

Irinotecan is a prodrug that is hydrolyzed to its active metabolite, SN-38, by hepatic carboxylesterases. Irinotecan is a semisynthetic derivative of the natural alkaloid camptothecin, which interacts with topoisomerase I and as a result exerts a cytotoxic effect. Topoisomerase I is involved in the uncoiling of DNA for replication and transcription and it causes single-stranded DNA breaks. Camptothecin stabilizes these breaks so they do not get repaired and this leads to DNA fragmentation and cell death through interaction with the replication fork.

Oxaliplatin is a platinum compound that forms cross-linking DNA adducts and consequent blocking of DNA replication and transcription. As a single agent oxaliplatin has almost no activity.

Bevacizumab is a humanized monoclonal antibody that binds to vascular endothelial growth factor-A (VEGF-A). VEGF-A is a central regulator of normal and tumor-associated angiogenesis. Binding to this growth factor leads to an inhibition in blood-vessel formation. Other anti-neoplastic effects of bevacizumab are attributed to normalization of grossly abnormal tumor vasculature, reduced intratumoral hydrostatic pressure and increased vessel leakiness, which allow bevacizumab and other agents to enter the tumor more effectively.

Cetuximab is a chimeric (human/mouse) monoclonal antibody that binds to the extracellular domain of the epidermal growth factor receptor (EGFR), and thereby blocks ligand-induced receptor signaling. Panitumumab is a fully human monoclonal antibody to EGFR and it acts similar to cetuximab.

Sphingolipids are lipids which are utilized by the cell for the regulation of the fluidity and the sub-domain structure of the lipid bilayers. Sphingolipids are complex lipids composed of a hydrophilic head group and a lipophilic backbone, which are derived from the aliphatic amino alcohol sphingosine, as depicted hereinbelow.

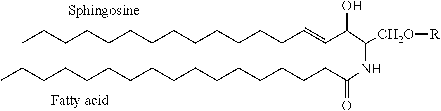

In ceramide, R═H;

In sphingomyelin, R=phosphocholine;

In glycosphingolipids (such as cerebrosides), R=sugar.

Within the past decade, progress has been made in understanding how sphingolipids contribute to disease processes, leading to potential novel therapeutic approaches based on interventions in sphingolipids homeostasis. Some of the areas in which particularly important advances have been made are cancer, sphingolipid storage diseases, immune diseases, cystic fibrosis, inflammation, emphysema, diabetes, sepsis, cardiovascular and neurological diseases.

Level changes of ceramide (the lipid component that makes up sphingomyelin) and/or sphingosine-1-phosphate are implicated in various stages of cancer pathogenesis and therapy. These include apoptosis, cell proliferation, cell migration, senescence and inflammation. Ceramide and its metabolites have important roles in regulating these processes. Ceramide mediates the regulation of growth arrest, senescence and apoptosis. When ceramide is generated in the plasma membrane it activates pathways associated with growth inhibition, oxidative stress-mediated cell death and lipid raft functions. When ceramide is generated in the endoplasmic reticulum (ER) it might be topologically associated with the nucleus.

Ceramide has a number of nuclear targets. It activates protein phosphatase-1 so as to dephosphorylate serine/arginine-rich proteins. Consequently, alternative splicing of pro-apoptotic proteins Bcl-XS or caspase-9 occurs. Another target of ceramide in the nucleus is a pro-survival protein telomerase, which catalyzes the elongation/maintenance of telomeres at the end of chromosomes. Recently, it has been suggested that endogenous ceramides with different fatty-acid chain lengths might have dissimilar roles in the cells.

One of the metabolic pathways of ceramide is to sphingosine by ceramidases and then to sphingosine-1-phosphate by sphingosine kinases. Sphingosine-1-phosphate is a pro-survival lipid, which plays a role in malignant transformation, cancer proliferation, inflammation, vasculorogenesis and resistance to apoptotic cell death. Sphingosine kinase 1 and sphingosine-1-phosphate influence the colon carcinogenesis, in part, by regulating COX-2 expression and prostaglandin E2 production. Ceramide is highly metabolized into glucosylceramide in some cancer cells due to an increase in glucosylceramide synthase activity and/or expression.

A mechanistic link between glucosylceramide synthase and P-glycoprotein, which is associated with drug resistance has been revealed.

Sphingomyelin is another metabolite of ceramide that is synthesized by sphingomyelin synthase. In studies conducted with mice, it has been found that dietary supplementation with sphingomyelin increased the portion of tumors that are histologically characterized as adenomas rather than the more malignant adenocarcinomas. Another study showed that consumption of glycosphingolipids suppresses colonic cell proliferation and aberrant crypt foci formation in 1,2-dimethylhydrazine-treated genetically-modified mice. These data suggest that different types of intervention in the metabolic pathways of ceramide may influence cancer development and therapy. Different approaches have been suggested for influencing sphingolipids metabolism, one of which relates to allyl alcohol derivatives, which are non-natural sphingolipid analogs, and which have been shown to act as efficient anticancer drugs [Raclin N. S., Bioorg Med Chem. 2003; 11:2123-42].

Boron is considered as an appealing pharmacore since it can mimic and thus inhibit many of the activities of the carboxy group. The use of boron-containing compounds to treat various cancers has until recently been restricted to Boron Neutron Capture Therapy (BNCT) [Soloway et al., Chem. Rev. 1998:98:515-62]. BNCT, however, requires a nuclear facility for practicing it and is therefore currently considered impractical.

In 2003, the first totally synthetic boron-containing compound has been introduced in the clinic as bortezomid (previously codenamed PS-341), known by its trademark Velcade®, for use against refractory and recurring myelomas [Yang et al., Curr. Prot. Pept. Sci., 2008:9:227-39]. Velcade inhibits proteasome, a protease that occurs in a much higher extent in certain cancerous cells. Velcade® is now a standalone treatment for blood myelomas, is also prescribed for treating relapsed mantle cells lymphoma (MCL), and has been for use in the treatment of other cancers.

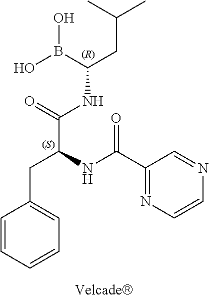

The present inventors have previously disclosed novel 3-hydroxy-1-alkenylboronates, prepared using phosphine stabilized borylzirconacyclopropenes [Quntar et al., Chem Commun, 2008:43:5589-91].

Additional background art includes Dembitsky et al. [Mini-Rev. Med. Chem., 2004:4:1001-18].

### SUMMARY OF THE INVENTION

The present inventors have now uncovered that 3-substituted vinylboronates exhibit an anti-cancer activity on various cell lines, presumably by modulating the metabolism of sphingolipids. The present inventors have also shown that these compounds are not protease inhibitors, i.e., their mode of action is different than that of Velcade®. This feature is highly advantageous in terms of the toxicity of the disclosed compounds.

It has been shown that 3-hydroxy-1-alkenylboronates reduce the survival of myeloma and colon cancer cells.

The use of highly substituted vinylboronates represents a new approach in colorectal cancer therapy and in cancer therapy in general.

According to an aspect of some embodiments of the present invention there is provided a method of treating cancer, the method comprising administering to a subject in need thereof a therapeutically effective amount of a 3-substituted vinylboronate.

According to an aspect of some embodiments of the present invention there is provided a use of a 3-substituted vinylboronate in the manufacture of a medicament.

In some embodiments, the medicament is for treating cancer.

According to an aspect of some embodiments of the present invention there is provided a pharmaceutical composition comprising a 3-substituted vinylboronate and a pharmaceutically acceptable carrier.

In some embodiments, the pharmaceutical composition is packaged in a packaging material and identified in print, in or on said packaging material, for use in the treatment of cancer.

According to an aspect of some embodiments of the present invention there is provided a compound comprising a 3-substituted vinylboronate, the compound being identified for use in the treatment of cancer.

According to an aspect of some embodiments of the present invention there is provided a method of modulating sphingolipid metabolism in cancer cells, the method comprising contacting the cells with an effective amount of a 3-substituted vinylboronate. In some embodiments, modulating sphingolipid metabolism comprises decreasing a level of sphingomyelin in cancer cells. In some embodiments, modulating sphingolipid metabolism comprises increasing ceramide levels and decreasing the levels of sphingomyelin and glucocerebroside in cancer cells.

In some embodiments, the compounds utilized in the various aspects of embodiments of the invention advantageously do not exhibit protease inhibition.

In some embodiments, the compounds, methods, compositions and uses described herein are for treating colorectal cancer.

In some embodiments, the compounds described herein are utilized in any of the methods, compositions and uses described herein in combination with an additional therapeutically active agent. In some embodiments, the compounds described herein and the additional therapeutically active agent act in synergy. In some embodiments, the additional therapeutically active agent is an anti-cancer agent. In some embodiments, it is an anti-cancer agent useful in the treatment of colorectal cancer.

Exemplary 3-substituted vinylboronates include, but are not limited to, 3-hydroxy vinylboronates, 3-amino vinylboronates, 3-amido vinylboronates and 3-carboxy vinylboronates. In some embodiments, the 3-substituted vinylboronate is a 3-hydroxy vinylboronate.

The present inventors have devised and successfully practiced a convenient synthetic route for preparing 3-substituted vinylboronates. Using this synthetic route, versatile 3-substituted vinylboronates have been prepared.

In some embodiments, 3-substituted vinylboronates according to the present embodiments are collectively represented by the general Formula I:

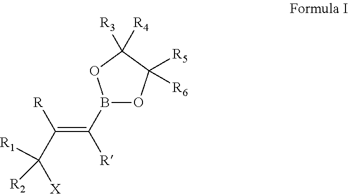

wherein:

X is selected from the group consisting of hydroxy, amine, amide, carboxy, thiocarboxy, thiol, alkoxy, thioalkoxy, aryloxy, thioaryloxy, sulfonamide, thioamide, carbamate, thiocarbamate, sulfonate, heteroalicyclic, heteroaryl, guanidinyl and guanyl, each can be substituted or unsubstituted, as defined herein;

R is selected from the group consisting of hydrogen, alkyl, alkenyl, cycloalkyl, aryl, heteroaryl, heteroalicyclic, alkoxy, aryloxy, thioalkoxy, thioaryloxy, alkylamino, aminoalkyl and the like, with R being either cis or trans to the boronate, and preferably cis;

R′ is hydrogen, although other substituents are also contemplated;

R₁ and R₂ are each independently selected from the group consisting of hydrogen, alkyl, alkenyl, alkynyl, cycloalkyl, aryl, heteroaryl, and heteroalicyclic, or alternatively, R₁ and R₂ form a 4-, 5- or 6-membered saturated or unsaturated, alicyclic or heteroalicyclic ring, optionally fused to another ring, while each of the above substituents can be further substituted or be unsubstituted, as defined herein; and

R₃-R₆ are each independently selected from the group consisting of alkyl, cycloalkyl, and aryl, or, alternatively, two of R₃-R₆ form a 4-, 5- or 6-membered saturated or unsaturated, alicyclic or heteroalicyclic ring.

In some embodiments, X is selected from the group consisting of hydroxy, amine, amide and carboxy.

In some embodiments, X is hydroxy.

In some embodiments, R is alkyl. In exemplary embodiments, R is selected from the group consisting of octyl, butyl and pentyl.

In some embodiments, each of R₃-R₆ is alkyl.

In some embodiments, each of R₃-R₆ is methyl.

In some embodiments, R₁ and R₂ are each independently selected from the group consisting of alkyl, cycloalkyl and aryl.

In some embodiments, R₁ is cyclopropyl.

In some embodiments, R₂ is phenyl.

In some embodiments, R₁ and R₂ form an alicyclic ring.

According to an aspect of some embodiments of the present invention, there are provided novel 3-substituted vinylboronate compounds, which can be collectively represented by the general Formula I as described herein, wherein at least of R, R₁ and R₂ is an alkyl being at least 6 carbon atoms in length.

In some embodiments of the present invention, R is alkyl.

In some embodiments of the present invention, R is said alkyl being at least 6 carbon atoms in length.

In some embodiments of the present invention, R₁ and R₂ are each independently selected from the group consisting of alkyl, cycloalkyl and aryl.

In some embodiments of the present invention, at least one of R₁ and R₂ is said alkyl being at least 6 carbon atoms in length.

In some embodiments of the present invention, at least one of R₁ and R₂ is a cycloalkyl.

In some embodiments of the present invention, R₁ is cyclopropyl.

In some embodiments of the present invention, R₂ is phenyl.

Exemplary novel 3-hydroxy vinylboronate include Compounds E5, E7 and E8 (see, FIG. 1).

According to an aspect of some embodiments of the present invention, there are provided novel 3-substituted vinylboronate compounds, which can be collectively represented by the general Formula II:

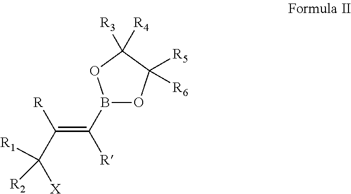

wherein:

X is selected from the group consisting of amine, amide, carboxy, thiocarboxy, thiol, alkoxy, thioalkoxy, aryloxy, thioaryloxy, sulfonamide, thioamide, carbamate, thiocarbamate, sulfonate, heteroalicyclic, heteroaryl, guanidinyl and guanyl, and all other variables are as defined herein.

In some embodiments, the novel 3-substituted vinylboronates having general Formula I or II are identified for use in the treatment of cancer.

According to an aspect of some embodiments of the present invention there is provided a pharmaceutical composition comprising the novel 3-substituted vinylboronates having general Formula I or II and a pharmaceutically acceptable carrier.

In some embodiments, the pharmaceutical composition is packaged in a packaging material and identified in print, in or on said packaging material, for use in the treatment of cancer, as described herein.

According to an aspect of some embodiments of the present invention there is provided a use of the novel compounds having general Formula I or II as a medicament.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, to exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents the 2D chemical structure of the exemplary 3-hydroxy vinylboronates according to some embodiments of the present invention;

FIGS. 2A-C present the effect of the exemplary 3-hydroxy vinylboronates according to some embodiments of the present invention Compounds A5 (FIG. 2A) and E1 (FIG. 2B) on ARH-77 cancer cells viability and of exemplary Compound A7 on HT-29 cancel cells viability;

FIGS. 3A-C are bar graphs showing the effect of exemplary 3-hydroxy vinylboronate Compound E1 on sphingolipid metabolism by demonstrating the effect of incubating ARH-77 cells in the presence of Compound E1 at the indicated concentrations on glucocerbroside (GC), ceramide and sphingomyelin (SPG) (FIG. 3A) and on GC and SPG (FIG. 3B) and the effect of incubating HT-29 cells in the presence of Compound E1 at the indicated concentrations on glucocerbroside (GC), ceramide and sphingomyelin (SPG) (FIG. 3C);

FIG. 4 is a bar graph showing the percents of SPM formation after 72 hours incubation with 0.5 μM/well BodiPy-12-Cer in A2780, A2780cisR, HT-29 and CRL-5803 cell-lines (10,000 cells/well). Data are presented from 3 independent triplicate experiments; and

FIGS. 5A-D are bar graphs demonstrating the lack of inhibitory activity of Compound E1 on the proteases Trypsin (FIG. 5A), Elastase (FIG. 5B), alpha-Chymotrypsin (FIG. 5C) and Leucine aminopeptidase (FIG. 5D).

### DESCRIPTION OF SPECIFIC EMBODIMENTS OF THE INVENTION

The present invention, in some embodiments thereof, relates to therapy and more particularly, but not exclusively, to a novel methodology for treating cancer such as colorectal cancer, which utilize boron-containing compounds.

The principles and operation of some embodiments of the present invention may be better understood with reference to the figures and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have now uncovered a novel approach to cancer therapy, which utilizes vinylboronate compounds.

More specifically, the present inventors uncovered that previously described 3-hydroxy vinylboronates [Quntar et al., Chem Commun, 2008:43:5589-91] are highly efficacious in reducing cancer cell viability. In a search for additional derivatives of vinylboronate compounds, the present inventors have designed a novel methodology for synthesizing other 3-substituted vinylboronate compounds and have prepared and successfully practiced novel 3-hydroxy vinylboronates. The present inventors have postulated that 3-substituted vinylboronate act by modulating the metabolism of sphingolipids, without exerting protease inhibition (unlike the currently known boron-containing drug Velcade). The compounds disclosed herein are therefore promising candidates for non-toxic cancer therapy.

According to an aspect of some embodiments of the present invention there is provided a method of treating cancer, the method comprising administering to a subject in need thereof a therapeutically effective amount of a 3-substituted vinylboronate.

According to an aspect of some embodiments of the present invention there is provided a use of a 3-substituted vinylboronate in the manufacture of a medicament.

In some embodiments, the medicament is for treating cancer.

According to an aspect of some embodiments of the present invention there is provided a compound comprising a 3-substituted vinylboronate, the compound being identified for use in treating cancer.

As used herein, the term “vinylboronate” describes a RaB(ORb)(ORc) moiety, in which Ra is an alkene, as defined herein, and Rb and Rc can be, for example, alkyl, aryl, cycloalkyl, and the like, or can be joined together to form a heteroalicyclic ring. This phrase therefore encompasses an ester of boronic acid, which is substituted by an α,β-unsaturated moiety.

The boronate can be either linear or cyclic. In some embodiments, Rb and Rc are joined together so as to form a cyclic boronate. The cyclic boronate can include a 4-, 5-, 6- or 7-membered ring, preferably having an alkylene bridge linking the two oxygen atoms. In some embodiments, the cyclic boronate is a 5-membered ring boronate, with an ethylene bridge linking the two oxygen atoms. In some embodiments, this ethylene bridge is substituted, as described hereinafter.

Exemplary 3-substituted vinylboronates include, but are not limited to, 3-hydroxy vinylboronates, 3-amino vinylboronates, 3-amido vinylboronates and 3-carboxy vinylboronates. In some embodiments, the 3-substituted vinylboronate is a 3-hydroxy vinylboronate.

The present inventors have devised and successfully practiced a convenient synthetic route for preparing 3-substituted vinylboronates, as is detailed in the Examples section that follows and is further described in Quntar et al. (2008, supra). Using this synthetic route, versatile 3-substituted vinylboronates have been prepared, and exemplary compounds were tested for anti-cancer activity and were shown highly effective in reducing viability of cancer cells.

In some embodiments, 3-substituted vinylboronates according to embodiments of the present invention are collectively represented by the general Formula I:

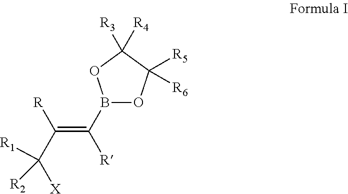

wherein:

X is selected from the group consisting of hydroxy, amine, amide, carboxy, thiocarboxy, thiol, alkoxy, thioalkoxy, aryloxy, thioaryloxy, sulfonamide, thioamide, carbamate, thiocarbamate, sulfonate, heteroalicyclic, heteroaryl, guanidinyl and guanyl, each can be substituted or unsubstituted, as defined herein;

R is selected from the group consisting of hydrogen, alkyl, alkenyl, cycloalkyl, aryl, heteroaryl, heteroalicyclic, alkoxy, aryloxy, thioalkoxy, thioaryloxy, alkylamino, aminoalkyl and the like, each can be substituted or unsubstituted, as described herein;

R′ is hydrogen, although other substituents are also contemplated;

R₁ and R₂ are each independently selected from the group consisting of hydrogen, alkyl, alkenyl, alkynyl, cycloalkyl, aryl, heteroaryl, and heteroalicyclic, or alternatively, R₁ and R₂ form a 4-, 5- or 6-membered saturated or unsaturated, alicyclic or heteroalicyclic ring, optionally fused to another ring, while each of the moieties defining R₁ and R₂ can be further substituted or be unsubstituted, as defined herein; and

R₃-R₆ are each independently selected from the group consisting of alkyl, cycloalkyl, and aryl, or, alternatively, two of R₃-R₆ form a 4-, 5- or 6-membered saturated or unsaturated, alicyclic or heteroalicyclic ring.

While other substituents are also contemplated for R₃-R₆, it is to be noted that substituents that do not include a heteroatom beta to the oxygen atoms of the boronate moiety are preferred.

In some embodiments, each of R₃-R₆ is independently an alkyl, preferably a lower alkyl such as methyl or ethyl.

In some embodiments, each of R₃-R₆ is methyl.

X in Formula I hereinabove represents a substituent at the allylic position: position 3 of the α,β-unsaturated moiety substituting the boronate.

In some embodiments, X is hydroxyl, such that the 3-substituted vinylboronate is 3-hydroxy vinylboronate.

Optionally, X is an amine, such that the 3-substituted vinylboronate is 3-amino vinylboronate.

Further optionally, X is an amide, such that the 3-substituted vinylboronate is 3-amido vinylboronate.

Further optionally, X is carboxy, such that the 3-substituted vinylboronate is 3-carboxy vinylboronate.

In some embodiments, X is an electron-donating group such as hydroxyl, thiol, alkoxy, thioalkoxy, aryloxy, thioaryloxy or amine.

Alternatively, X is an electron-withdrawing group such as amide and carboxy.

It is noted that in embodiments where X is hydroxy, the 3-substituted vinylboronate resembles a structure of a sphingolipid, the latter possessing an allylic hydroxyl.

The allylic carbon at position 3 of the vinylboronate, which bears the X substituent, can be either cis or trans to the boronate.

In some embodiments, the allylic carbon at position 3 which bears the X substituent is trans to the boronate.

The other substituent of the α,β-unsaturated moiety substituting the boronate is represented by the variable R in Formula I hereinabove.

R can be either cis or trans to the boronate, and is preferably cis.

In some embodiments, R is alkyl. In some embodiments, R is a medium alkyl having at least 2, at least 3 or at least 4 carbon atoms in its backbone chain. In some embodiments, the alkyl has 4-20 carbon atoms in its backbone chain. Higher and lower alkyls are also contemplated. In exemplary embodiments, R is n-octyl. Optionally, R is n-butyl or n-pentyl.

Alternatively, R can be an aryl (e.g., phenyl).

Without being bound by any particular theory, it is suggested that the moiety represented by the variable R in general Formula I herein should resemble the alkylene chain in the sphingosine moiety of a sphingolipid. Accordingly, in some embodiments, R is a hydrophobic moiety such as an alkyl as described hereinabove or an aryl.

It is to be noted that the nature of the moiety represented by R in Formula I herein is determined by the alkyne used as the starting material in the synthesis of the 3-substituted vinylboronates. Thus, versatile groups can be selected as this moiety and can be successfully used in preparing the final 3-substituted vinylboronate.

The moieties represented by variables R₁ and R₂ in Formula I described herein represent additional substituents on the allylic position.

In some embodiments, one or both R₁ and R₂ comprise a hydrophobic moiety. Without being bound by any particular theory, it is suggested when R₁ and/or R₂ is hydrophobic, structures analogous to sphingolipids are obtained.

Thus, in some embodiments, one or both, preferably both, R₁ and R₂ is independently a hydrophobic moiety such as alkyl, cycloalkyl or aryl.

In some embodiments, whenever R₁ and/or R₂ is an alkyl, the alkyl is preferably a medium alkyl, as described hereinabove for the variable R.

In some embodiments, one or both R₁ and R₂ comprise a cyclic moiety such as cycloalkyl and/or aryl, as there terms are defined herein.

Alternatively, the cyclic moiety can be a heteroaryl or a heteroalicyclic.

In some embodiments, R₁ is a cycloalkyl, for example, cyclopropyl. Higher cycloalkyls are also contemplated. It is to be noted that the size of the cycloalkyl may affect both the synthesis of the 3-substituted vinylboronate and its activity. Accordingly, cycloalkyls comprising 3, 4, 5 or 6 carbons atoms within the ring may be considered as preferred, with lower cycloaklkyls may be considered as most preferred.

In some embodiments, R₂ is aryl, for example, phenyl. A presence of an aromatic moiety at the allylic position can provide beneficial electronic effects.

In some embodiments, R₁ is cycloalkyl (e.g., cyclopropyl) and R₂ is aryl (e.g., phenyl).

In some embodiments, R₁ and R₂ form together a ring. The ring can be aromatic or alicyclic.

In some embodiments, R₁ and R₂ form together an alicyclic ring (i.e., a cycloalkyl), such as, for example, cyclohexyl, cyclopentyl or cycloheptyl. Unsaturated alicyclic rings are also contemplated.

In some embodiments, the alicyclic ring comprises an aryl which can be a substituent of the cycloalkyl or can be fused thereto. Substitution or fusion can be at any position of the alicyclic ring. In some embodiments, the substitution or fusion are at an ortho position with respect to the substituent at position 3 of the vinylboronate.

In some embodiments, R₁ and R₂ form together an alicyclic ring as defined herein, fused to an aromatic ring (namely, has an aryl ring fused thereto).

In some embodiments, R₁ and R₂ form together a cycloalkyl fused to phenyl (e.g., a tetrahydronaphthalene).

R₁ and R₂ in the general Formula described hereinabove can be determined by the nature of the ketone or aldehyde reactant used for preparing the vinyl boronate compounds (see, the Examples section that follows). Reactants such as acetone, benzaldehyde, anisaldehyde, cyclopropylphenyl ketone, tetralone and cyclohexanone were all shown to successfully form a 3-hydroxy vinyl boronate (see, Quntar et al. 2008, supra), which can be further reacted so as replace the hydroxyl substituent at position 3 by other substituents as exemplified in the Examples section hereinbelow.

Without being bound by any particular theory, it is suggested that the nature of R₁ and R₂ is selected such that (i) the ketone or aldehyde reactant is compatible with the reaction conditions, namely, will account for a successful synthesis of a corresponding intermediate (see, for example, Scheme 1 in the Examples section that follows); and (ii) will provide the compound with desired characteristics for exhibiting an anti-cancer effect, presumably by forming a structure that is analogous to sphingolipid, as described hereinabove.

Thus, further without being bound by any particular theory, while hydrophobicity is required for successful activity, it may be suggested that groups that are less bulky, such as cyclopropyl and aryl, may allow both successful synthesis and successful performance in terms of intervention with sphingolipid metabolism due to a less bulky structure.

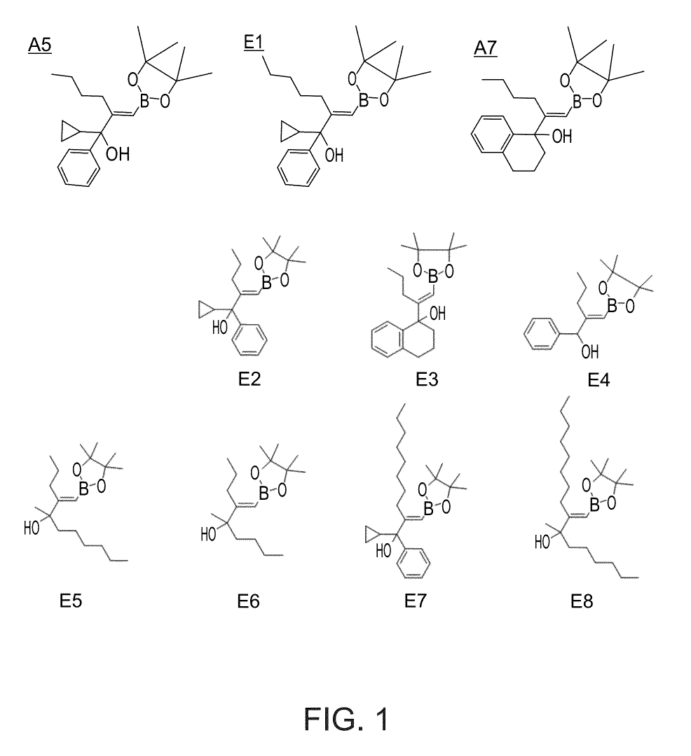

Exemplary 3-substituted vinylboronates are presented in FIG. 1 and further in the Examples section that follows.

In some embodiments, the 3-substituted vinylboronate is any one of Compounds E1, E2, E3, A5, A7, E5, E6 and E7, as depicted in FIG. 1.

In some embodiments, the 3-substituted vinylboronate is Compounds E7, as depicted in FIG. 1.

As used herein, the term “cancer” encompasses a class of diseases in which a group of cells display uncontrolled growth (division beyond the normal limits). The term “cancer” encompasses malignant and benign tumors as well as disease conditions evolving from primary or secondary tumors.

The term “malignant tumor” describes a tumor which is not self-limited in its growth, is capable of invading into adjacent tissues, and may be capable of spreading to distant tissues (metastasizing). The term “benign tumor” describes a tumor which is not to malignant (i.e. does not grow in an unlimited, aggressive manner, does not invade surrounding tissues, and does not metastasize). The term “primary tumor” describes a tumor that is at the original site where it first arose. The term “secondary tumor” describes a tumor that has spread from its original (primary) site of growth to another site, close to or distant from the primary site.

Cancers treatable by the compounds described herein include, but are not limited to, solid tumors, including carcinomas, and non-solid tumors, including hematologic malignancies. Carcinomas include, but are not limited to, adenocarcinomas and epithelial carcinomas. Hematologic malignancies include leukemias, lymphomas, and multiple myelomas.

Non-limiting examples of the cancers treatable by the compounds described herein include ovarian, pancreas, brain, colon, rectal, colorectal, melanoma, lung, breast, kidney, and prostate cancers.

The term “cancer metastases” describes cancer cells which have “broken away”, “leaked”, or “spilled” from a primary tumor, entered the lymphatic and/or blood vessels, circulated through the lymphatic system and/or bloodstream, settled down and proliferated within normal tissues elsewhere in the body thereby creating a secondary tumor.

In some embodiments, the cancer treatable by the compounds described herein is colorectal cancer, colon cancer or rectal cancer.

In some embodiments, the cancer is myeloma (multiple myeloma cancer).

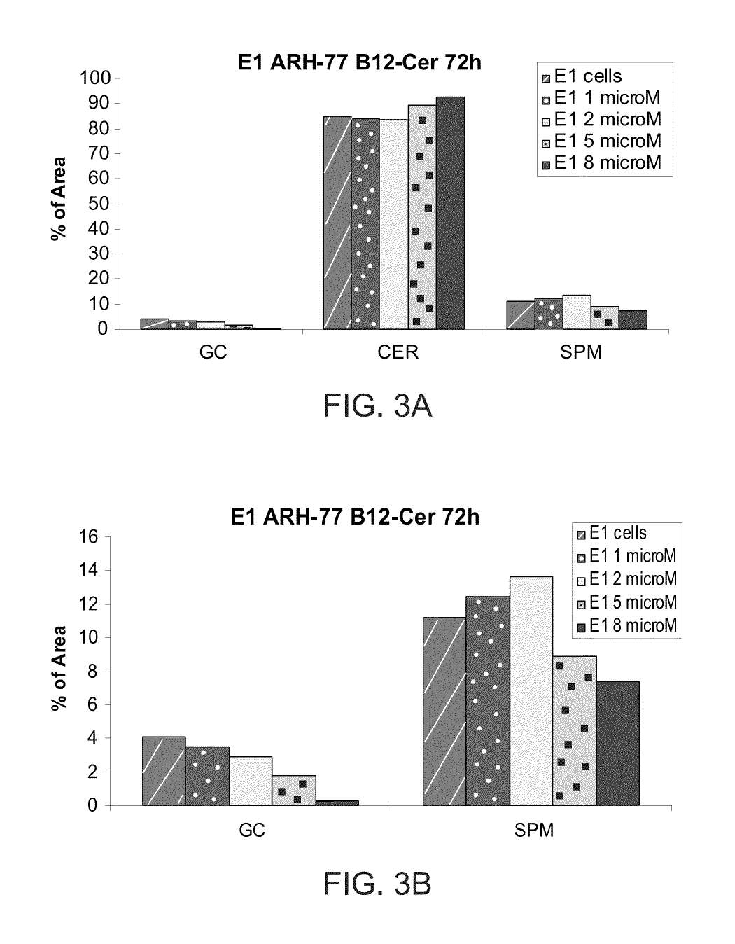

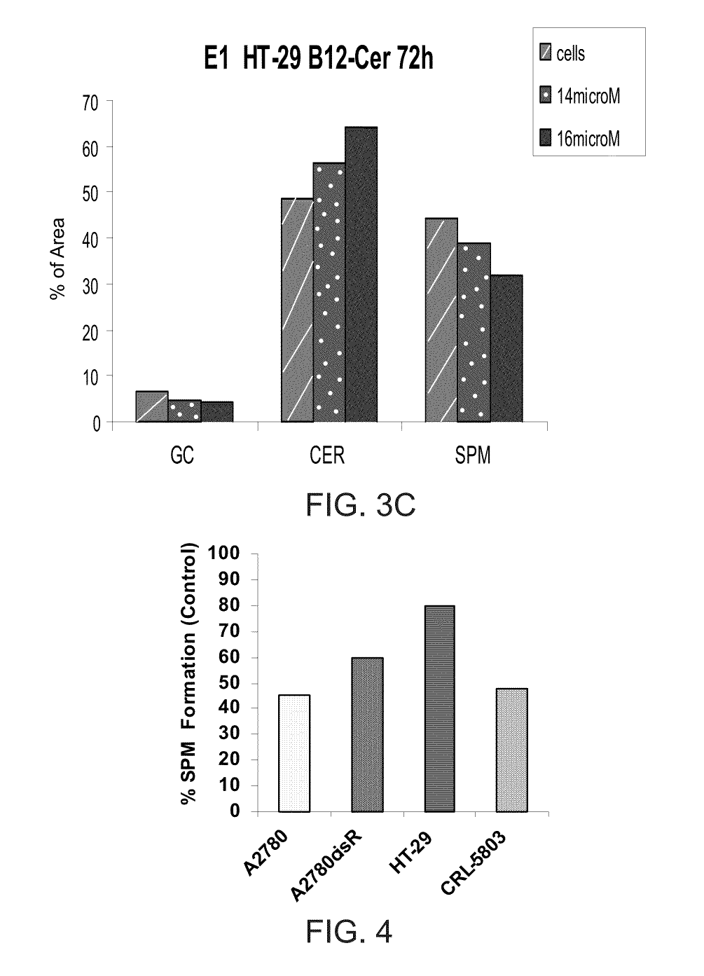

As demonstrated in the Examples section that follows, the present inventors have shown that 3-substituted vinylboronate affect sphingolipid metabolism in cancer cell lines, by increasing ceramide levels and decreasing levels of sphingomyelin and glucocerebroside (see, for example, Example 5 and FIGS. 3A-B).

The showing that shpingomyelin is synthesized to a higher level in colorectal cancer cell lines further supports a role for the 3-substituted vinylboronates described herein in treating this type of cancer.

According to an aspect of some embodiments of the present invention there is provided a method of modulating sphingolipid metabolism in cancer cells, the method comprising contacting the cells with an effective amount of a 3-substituted vinylboronate.

In some embodiments, modulating sphingolipid metabolism comprises increasing ceramide levels in cancer cells.

In some embodiments, modulating sphingolipid metabolism comprises decreasing a level of sphingomyelin in cancer cells.

In some embodiments, modulating sphingolipid metabolism comprises decreasing a level of glucocerebroside in cancer cells.

In some embodiments, modulating sphingolipid metabolism comprises both increasing ceramide levels and decreasing the levels of sphingomyelin and glucocerebroside in cancer cells.

As further demonstrated in the Examples section that follows, 3-substituted vinylboronate were assayed for their effect on various proteases and were found to be substantially devoid of protease inhibition activity. This feature allows using these compounds while avoiding the toxicity and related side effects that are associated with administration of protease inhibitors.

Thus, in some embodiments, the compounds utilized in the various aspects of embodiments of the invention advantageously do not exhibit protease inhibition.

In some embodiments, the compounds described herein are utilized in any of the methods, compositions and uses described herein in combination with an additional therapeutically active agent.

Exemplary additional therapeutically active agents that may act in synergy with the 3-substituted vinylboronate compounds described herein include, but are not limited to, anti-cancer agents, such as, but not limited to, chemotherapeutic agents, including alkylating agent, natural products such as taxanes, antibiotics, platinum-coordination complexes, hormones; anti-angiogenesis agents; radioactive agents; anti-inflammatory agents, anti-microbial agents, anti-depressant, analgesics, etc.

In some embodiments, the additional therapeutically active agent is an anti-cancer agent, as described herein. The anti-cancer agent in an agent useful in treating the cancer for which the 3-substituted vinylboronate compound is used to treat. A person skilled in the art would recognize those anti-cancer agents that are useful to treat each cancer type.

In some embodiments, the methods and uses described herein are for treating colorectal cancer and the anti-cancer agent is useful in the treatment of colorectal cancer.

Exemplary agents useful in the treatment of colorectal cancer include, but are not limited to, 5-fluorouracil (5-FU), capecitabine (Xeloda), Leucovorin (LV, folinic Acid), Oxaliplatin (Eloxatin), UFT or Tegafur-uracil, Irinotecan (Camptosar), Bevacizumab (Avastin), Cetuximab (Erbitux), Panitumumab (Vectibix), Bortezomib (Velcade), Oblimersen (Genasense, G3139), Gefitinib and erlotinib (Tarceva), and Topotecan (Hycamtin), as well as of the agents described hereinabove.

In some embodiments, the compounds described herein and the additional therapeutically active agent act in synergy. This beneficially allows using less than the recognized therapeutically effective amount of the anti-cancer agent.

In any of the methods and uses described herein, the 3-substituted vinylboronate can be utilized either per se, or, preferably as a part of a pharmaceutical composition which further comprises a pharmaceutically acceptable carrier.

As used herein a “pharmaceutical composition” refers to a preparation of one or more of the vinylboronates described herein, with other chemical components such as pharmaceutically acceptable and suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, Pa., latest edition, which is incorporated herein by reference.

Pharmaceutical compositions for use in accordance with the present invention thus may be formulated in conventional manner using one or more pharmaceutically acceptable carriers comprising excipients and auxiliaries, which facilitate processing of the vinylboronates into preparations which can be used pharmaceutically. Proper formulation is dependent upon the route of administration chosen. The dosage may vary depending upon the dosage form employed and the route of administration utilized. The exact formulation, route of administration and dosage can be chosen by the individual physician in view of the patient's condition (see e.g., Fingl et al., 1975, in “The Pharmacological Basis of Therapeutics”, Ch. 1 p. 1).

The pharmaceutical composition may be formulated for administration in either one or more of routes depending on whether local or systemic treatment or administration is of choice, and on the area to be treated. Administration may be done orally, by inhalation, or parenterally, for example by intravenous drip or intraperitoneal, subcutaneous, intramuscular or intravenous injection, or topically (including ophtalmically, vaginally, rectally, intranasally).

Formulations for topical administration may include but are not limited to lotions, ointments, gels, creams, suppositories, drops, liquids, sprays and powders. Conventional pharmaceutical carriers, aqueous, powder or oily bases, thickeners and the like may be necessary or desirable.

Compositions for oral administration may include, but are not limited to, powders or granules, suspensions or solutions in water or non-aqueous media, sachets, pills, caplets, capsules or tablets. Thickeners, diluents, flavorings, dispersing aids, emulsifiers or binders may be desirable.

Formulations for parenteral administration may include, but are not limited to, sterile solutions which may also contain buffers, diluents and other suitable additives. Slow release compositions are envisaged for treatment.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert.

Compositions comprising a 3-substituted vinylboronate as described herein, formulated in a compatible pharmaceutical carrier, may also be prepared, placed in an appropriate container, and labeled for treatment of a particular medical condition, as is detailed hereinabove.

According to some embodiments of the present invention, a pharmaceutical composition comprising a 3-substituted vinylboronate as described herein is packaged in a packaging material and is identified in print, in or on the packaging material, for use in the treatment of cancer, as described herein.

In some embodiments, the pharmaceutical composition is identified for use in combination with an additional therapeutically active agent, as described herein.

In some embodiments, the pharmaceutical composition further comprises an additional therapeutically active agent as described herein.

In the course of studying 3-substituted vinylboronates, the present inventors have devised various methodologies for preparing 3-substituted vinylboronates other than the previously described 3-hydroxy vinylboronates. Using such methodologies, novel 3-substituted vinylboronates can be prepared.

Thus, as discussed hereinabove, the present inventors have contemplated 3-substituted vinylboronates that comprise one or more hydrophobic moieties as resembling sphingolipids and thus as exhibiting enhanced anti-cancer activity, and have indeed shown that 3-substituted vinylboronate bearing a relatively long alkyl chain (e.g., a medium alkyl) as one or more of the substituents R, R₁ and R₂ is Formula I can be successfully prepared.

According to an aspect of some embodiments of the present invention there are to provided novel 3-substituted vinylboronate compounds having general Formula I as depicted hereinabove, in which at least one of R, R₁ and R₂ is an alkyl being at least 6 carbon atoms in length, at least 7 carbon atoms, or at least 8 carbon atoms in length.

In some embodiments, R is an alkyl being at least 6 carbon atoms in length (e.g., a 6-20 carbon atoms alkyl). In some embodiments, R is alkyl being 6, 7, 8, 9, 10, 11 and even 12 or more carbon atoms in length. In some embodiments, R is octyl, for example, n-octyl.

In some embodiments, R is said alkyl being at least 6 carbon atoms in length and R₁ and R₂ are as defined hereinabove.

In some embodiments, at least one of R₁ and R₂ is an alkyl being at least 6 carbon atoms in length. In some embodiments, one of R₁ and R₂ is an alkyl being at least 6 carbon atoms in length, and one of R₁ and R₂ is an alkyl such as methyl.

Exemplary such compounds include, but are not limited to, Compounds E5, E7 and E8 (see, for example, FIG. 1).

As further discussed hereinabove, the present inventors have also contemplated 3-substituted vinylboronates, in which the allylic group denoted as X in Formula I hereinabove, is other then hydroxy.

According to an aspect of some embodiments of the present invention, there are provided novel 3-substituted vinylboronate compounds, which can be collectively represented by the general Formula II:

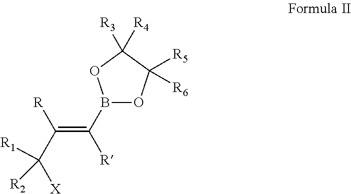

wherein:

X is selected from the group consisting of amine, amide, carboxy, thiocarboxy, thiol, alkoxy, thioalkoxy, aryloxy, thioaryloxy, sulfonamide, thioamide, carbamate, thiocarbamate, sulfonate, heteroalicyclic, heteroaryl, guanidinyl and guanyl, and all other variables are as defined hereinabove for compounds having general Formula I.

In some embodiments, the novel 3-substituted vinylboronates described herein (e.g., having general Formula I in which at least one of R, R₁ and R₂ is an alkyl having at least 6 carbon atoms or having general Formula II) are identified for use in the treatment of cancer.

According to an aspect of some embodiments of the present invention there is provided a pharmaceutical composition comprising the novel 3-substituted vinylboronates described herein (e.g., having general Formula I in which at least one of R, R₁ and R₂ is an alkyl having at least 6 carbon atoms or having general Formula II) and a pharmaceutically acceptable carrier, as defined herein.

In some embodiments, the pharmaceutical composition is packaged in a packaging material and identified in print, in or on said packaging material, for use in the treatment of cancer, as described herein.

According to an aspect of some embodiments of the present invention there is provided a use of the novel 3-substituted vinylboronates as described herein (e.g., having general Formula I in which at least one of R, R₂ and R₂ is an alkyl having at least 6 carbon atoms or compound having general Formula II) as a medicament.

In some embodiments, the medicament is for use in the treatment of cancer (e.g., colorectal cancer), as described herein.

According to an aspect of some embodiments of the present invention there is provided a method of treating cancer, as described herein, which is effected by administering to a subject in need thereof a therapeutically effective amount of the novel 3-substituted vinylboronates as described herein (e.g., having general Formula I in which at least one of R, R₁ and R₂ is an alkyl having at least 6 carbon atoms or compounds having general Formula II).

As used herein, the term “amine” describes both a —NR′R″ group and a —NR′— group, wherein R′ and R″ are each independently hydrogen, alkyl, cycloalkyl, aryl, as these terms are defined hereinbelow.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine.

The term “alkyl” describes a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms, or 4 to 10 carbon atoms, or 6 to 10 carbon atoms. The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, to O-carbamate, C-amide, N-amide, guanyl, guanidine and hydrazine.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. Representative examples are piperidine, piperazine, tetrahydrofurane, tetrahydropyrane, morpholino and the like.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. Representative examples are pyridine, pyrrole, oxazole, indole, purine and the like.

The term “halide” and “halo” describes fluorine, chlorine, bromine or iodine.

The term “haloalkyl” describes an alkyl group as defined above, further substituted by one or more halide.

The term “carbonyl” or “carbonate” as used herein, describes a —C(═O)—R′ group, with R′ as defined herein.

The term “thiocarbonyl” as used herein, describes a —C(═S)—R′ group, with R′ as defined herein.

The term “hydroxyl” describes a —OH group.

The term “alkoxy” describes both an —O-alkyl and an —O-cycloalkyl group, as defined herein.

The term “aryloxy” describes both an —O-aryl and an —O-heteroaryl group, as defined herein.

The term “thiohydroxy” describes a —SH group.

The term “thioalkoxy” describes both a —S-alkyl group, and a —S-cycloalkyl group, as defined herein.

The term “thioaryloxy” describes both a —S-aryl and a —S-heteroaryl group, as defined herein.

The term “cyano” describes a —C≡N group.

The term “isocyanate” describes an —N═C═O group.

The term “nitro” describes an —NO₂ group.

The term “acyl halide” describes a —(C═O)R″″ group wherein R″″ is halide, as defined hereinabove.

The term “azo” or “diazo” describes an —N═NR′ group, with R′ as defined hereinabove.

The term “carboxylate” encompasses “C-carboxylate”, which describes a —C(═O)—OR′ group, where R′ is as defined herein; and “O-carboxylate”, which describes a —OC(═O)R′ group, where R′ is as defined herein.

The term “thiocarboxylate” encompasses “C-thiocarboxylate”, which describes a —C(═S)—OR′ group, where R′ is as defined herein; and “O-thiocarboxylate”, which describes a —OC(═S)R′ group, where R′ is as defined herein.

The term “carbamate” encompasses “N-carbamate”, which describes an R″OC(═O)—NR′— group, with R′ and R″ as defined herein; and “O-carbamate”, which describes an —OC(═O)—NR′R″ group, with R′ and R″ as defined herein.

The term “thiocarbamate” encompasses “O-thiocarbamate”, which describes a —OC(═S)—NR′R″ group, with R′ and R″ as defined herein; “N-thiocarbamate”, which describes an R″OC(═S)NR′— group, with R′ and R″ as defined herein; “S-dithiocarbamate”, which describes a —SC(═S)—NR′R″ group, with R′ and R″ as defined herein; and “N-dithiocarbamate”, which describes an R″SC(═S)NR′— group, with R′ and R″ as defined herein.

The term “urea”, which is also referred to herein as “ureido”, describes a —NR′C(═O)—NR″R′″ group, where R′ and R″ are as defined herein and R″′ is as defined herein for R′ and R″.

The term “thiourea”, which is also referred to herein as “thioureido”, describes a —NR′—C(═S)—NR″R′″ group, with R′, R″ and R″′ as defined herein.

The term “amide” encompasses “C-amide”, which describes a —C(═O)—NR′R″ group, where R′ and R″ are as defined herein; and “N-amide”, which describes a R′C(═O)—NR″— group, where R′ and R″ are as defined herein.

The term “guanyl” describes a R′R″NC(═N)— group, where R′ and R″ are as defined herein.

The term “guanidine” describes a —R′NC(═N)—NR″R″′ group, where R′, R″ and R″′ are as defined herein.

The term “hydrazine” describes a —NR′—NR″R′″ group, with R′, R″, and R″′ as defined herein.

The term “silyl” describes a —SiR′R″R″′ group, whereby each of R′, R″ and R′″ are as defined herein.

The term “siloxy” describes a —Si(OR′)R″R″′ group, whereby each of R′, R″ and R″′ are as defined herein.

The term “silaza” describes a —Si(NR′R″)R′″ group, whereby each of R′, R″ to and R″′ is as defined herein.

The term “silicate” describes a —O—Si(OR′)(OR″)(OR″′) group, with R′, R″ and R″′ as defined herein.

The term “sulfate” describes a —O—S(═O)₂—OR′ group, where R′ is as defined hereinabove.

The term “thiosulfate” describes a —O—S(═S)(═O)—OR′ group, where R′ is as defined hereinabove.

The term “sulfite” describes an —O—S(═O)—O—R′ group, where R′ is as defined hereinabove.

The term “thiosulfite” describes a —O—S(═S)—O—R′ group, where R′ is as defined hereinabove.

The term “sulfinate” describes a —S(═O)—OR′ group, where R′ is as defined hereinabove.

The term “sulfoxide” or “sulfinyl” describes a —S(═O)R′ group, where R′ is as defined hereinabove.

The term “sulfonate” describes a —S(═O)₂—R′ group, where R′ is as defined herein.

The term “S-sulfonamide” describes a —S(═O)₂—NR′R″ group, with R′ as defined herein and R″ is as defined herein for R′.

The term “N-sulfonamide” describes an R′S(═O)₂—NR″— group, where R′ and R″ are as defined herein.

The term “phosphonate” describes a —P(═O)(OR′)(OR″) group, with R′ and R″ as defined herein.

The term “thiophosphonate” describes a —P(═S)(OR′)(OR″) group, with R′ and R″ as defined herein.

The term “phosphinyl” describes a —PR′R″ group, with R′ and R″ as defined hereinabove.

The term “phosphite” describes an —O—PR′(═O)(OR″) group, with R′ and R″ as defined herein.

Any of the compounds described herein (3-substituted vinylboronates) can be in a form of a pharmaceutically acceptable salt thereof, prodrugs thereof, solvates or hydrates thereof.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound.

As used herein, the term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. The prodrug may also have improved solubility as compared with the parent drug in pharmaceutical compositions. Prodrugs are also often used to achieve a sustained release of the active compound in vivo.

The term “solvate” refers to a complex of variable stoichiometry (e.g., di-, tri-, tetra-, penta-, hexa-, and so on), which is formed by a solute (the 3-substituted vinylboronate, as described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

Isomers, including stereoisomers and regioisomers, of the compounds described herein, are also contemplated. Further contemplated are isomorphs of the compounds described herein.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be to construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the term “therapeutically effective amount” denotes that dose of an active ingredient or a composition comprising the active ingredient that will provide the therapeutic effect for which the active ingredient is indicated, herein treating cancer.

In some embodiments, the therapeutically effective amount ranges from 0.01 mg/kg body to 100 mg/kg body.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Syntheses of 3-hydroxyvinylboronates

A series of 3-Hydroxy vinylboronates were prepared as described in Quntar et al., Chem Commun, 2008:43:5589-91, by reacting 1-2-(hex-1-ynyl)-4,4,5,5-tetramethyl-1,3,2-dioxaborolane with the Negishi reagent (Cp₂ZrCl₂/2n-BuLi) (while stabilizing the intermediate zirconacyclopropene boronate with phosphine ligands, and thereafter adding an aldehyde or ketone to the stabilized intermediate so as to furnish the desired 3-hydroxy vinylboronates.

An exemplary general synthesis is presented in Scheme 1 below.

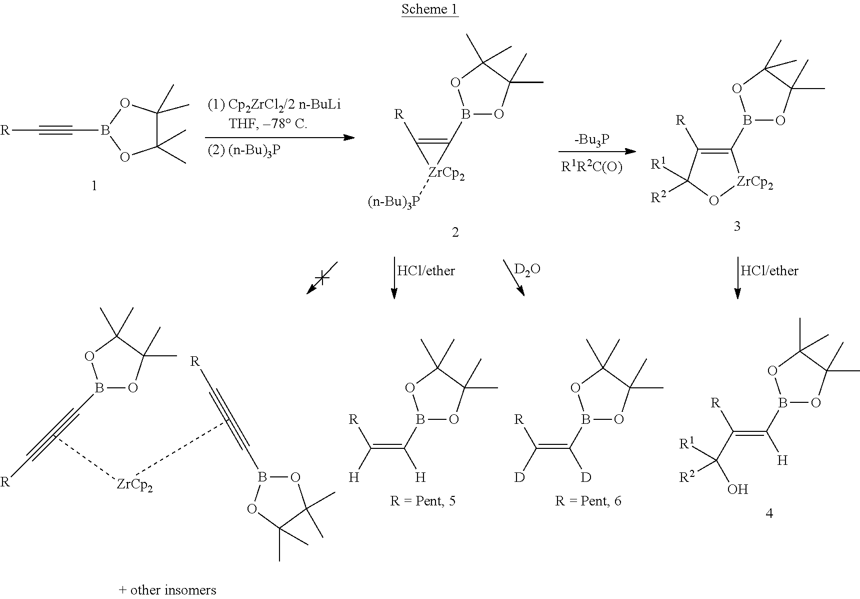

The allylic alcohol moiety of these compounds renders them structurally similar to sphingolipid analogs that are known to induce apoptotic cell death.

Using the above-described synthetic pathway, 3-hydroxy vinylboronates have been prepared having various substituents as variables R, R₁ and R₂ were prepared.

As noted in Scheme 1 above, the nature of the variable R can be determined by selecting the alkyne used as the starting material (see, Compound 1, Scheme 1). The nature of variables R₁ and R₂ can be determined by selecting the ketone or aldehyde reactant used to form the intermediate Compound 3 in Scheme 1. The selected ketone should be such that would be compatible with the reaction for forming intermediate Compound 3.

The structures of exemplary 3-hydroxy vinylboronates, according to some embodiments of the invention, in which the variables R, R₁ and R₂ were selected so as to resemble sphingolipid structures, are presented in FIG. 1.

### Example 2

### Syntheses of 3-Amino/amido vinylboronates

Amino/amido boronates possess significant anticancer activity [Dembitsky et al., Mini-Rev. Med. Chem., 2004:4:1001-18].

3-Aminovinylboronates are prepared from 3-hydroxy vinylboronates by a modification of the Mitsunobu reaction [Mitsunobu O., Synthesis, 1981:1-28] as presented in Scheme 2 below.

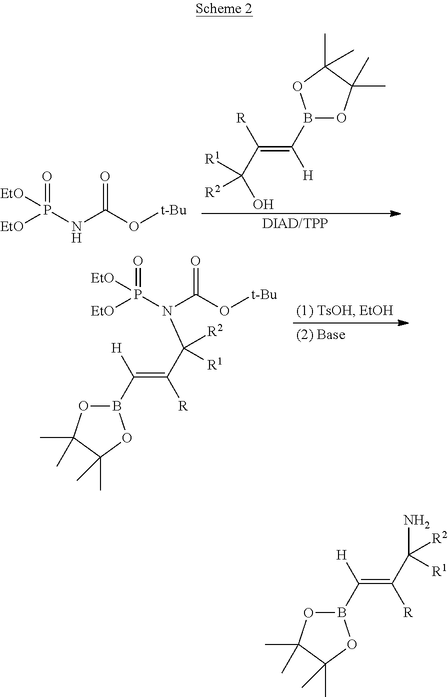

3-Hydroxy vinylboronates are reacted with N-(t-butoxycarbonyl)phosphoramidate in the presence of diisopropylazodicarboxylate (DIAD) and triphenylphosphine (TPP) followed by treating the diethyl N-alkyl-N-(t-butoxycarbonyl)phosphoramidates briefly with p-toluenesulfonic acid, then carefully basifying the reaction mixture [Klepacz A. and Zwierzak A., Synth. Commun., 2001:31(11):1683-89].

3-Amido vinylboronates are obtained by a Mitsunobu reaction of the 3-hydroxy vinylboronates with a cyclic/acyclic amide followed by mild hydrolysis [Mitsunobu, O. and Wada, M., J. Am. Chem. Soc., 1972; 94:679-80], as presented in Scheme 3 below.

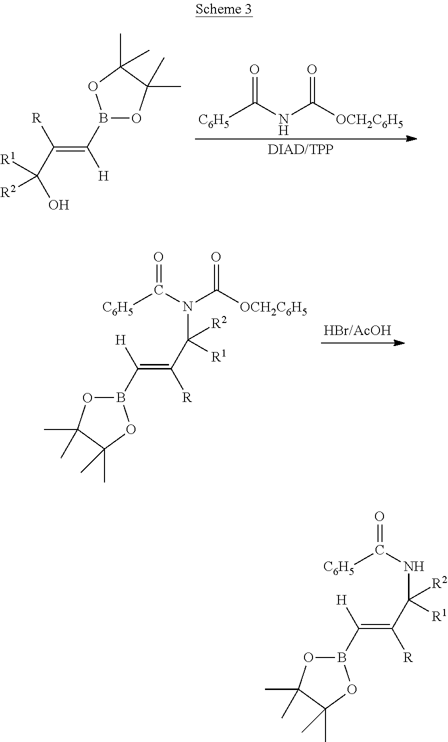

### Example 3

### Syntheses of 3-Acyloxy vinylboronates

3-Acyloxy vinylboronates are similarly synthesized by a Mitsunobu reaction of 3-hydroxy vinylboronates and a carboxylic acid, as presented in Scheme 4 below.

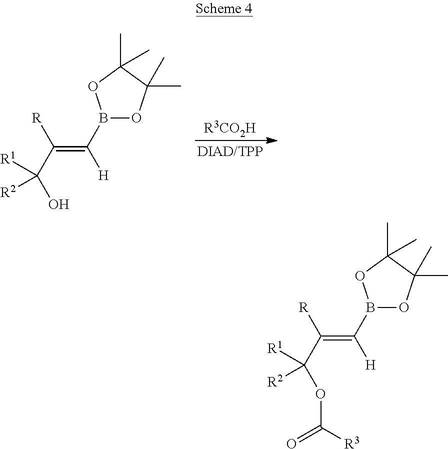

Comparing the activity of 3-acyloxy vinylboronates and 3-amine/amide vinylboronates to that of 3-hydroxy vinylboronates is used for determining the effect of an electron donating group such as a free hydroxyl group on anticancer activity.

### Example 4

### Activity Assays

MTT Assay:

ARH-77 (myeloma) and HT-29 (colon cancer) cells were grown and counted, and were thereafter plated in 96-wells plates to get 0.1×10⁶ cells/well. The plates were left in the incubator overnight for cells attachment and the tested compounds were then added to the cells. The tested concentrations of the compounds were 1, 2, 5, 10, 20, 50 and 100 μM. The compounds were incubated with the cells for 24 hours, 48 hours or for 72 hours, and were thereafter subjected to the MTT assay.

FIG. 1 presents the chemical structures of the 3-hydroxy vinylboronates that were tested, which are denoted herein as A5, E1 and A7.

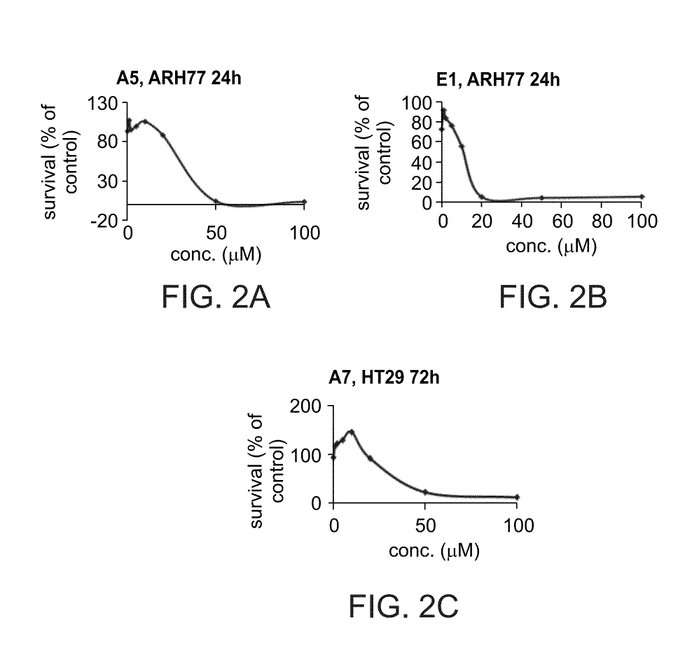

FIGS. 2A-C presents some of the data obtained for the tested compounds.

As can be seen in FIGS. 2A-C, all tested compounds exhibited a substantial effect in reducing cancer cells survival.

Table 1 below presents the data obtained for additional 3-hydroxy vinylboronates. Data presents the IC₅₀ values recorded.

TABLE 1

| | Cell type | |
|---|---|---|
| | | ARH-77, 72 h incubation, |
| Compound | HT-29, 72 h incubation, μM | μM |
| E1 | not active | 11 |
| E2 | 85 | 54 |
| E3 | 85 | 65 |
| E4 | >100 | not active |
| E5 | 55 | 30 |
| E6 | 80 | 54 |
| E7 | 15 | 6 |
| E8 | >100 | 78 |

As can be seen in Table 1, while most of the tested compounds affected cancer cell growth, a more pronounced activity is observed in compounds having substituents with higher hydrophobicity.

Fluorescence Activated Cell Sorter (FACS):

Cell cultures are incubated with the tested compounds as described hereinabove and are thereafter stained with propidium iodide. Then, by using the FACS, the sub-G1 peak is detected.

Annexin V:

Cells apoptosis is accompanied by the expression of phosphatidyl serine at the cells surface. This expression is important for macrophages that recognize and eliminate the apoptotic cells. Fluorescent labeled Annexin V is utilized for detecting cells that express phosphatidyl serine at their surface using FACS.

DNA-Fragmentation:

At the end of the apoptosis-signaling cascade particular endonucleases are activated and cut the chromosomal DNA. The DNA fragments are subjected to agarose gel electrophoresis and are detected with antibodies, or with a DNA stain.

Caspase:

Cell cultures are incubated with the tested compounds as described hereinabove and thereafter Caspase 3/7 or 9 activation is determined by activity assays using commercially available kits.

Synergism with Chemotherapeutic Drugs:

The potential synergistic effect of the 3-substituted vinylboronates with chemotherapeutic drugs is tested by co-administration of these agents to cancer cells. The cytotoxic effect of the combined treatment is measured by the MTT assay. The results are compared to the cytotoxic effect of each of the 3-substituted vinylboronates and each of the chemotherapeutic drugs alone.

In Vivo Studies:

The in vivo anticancer activity of 3-substituted vinylboronates is examined on mice. Colon cancer cells are injected to nude mice intradermally (i.d.) or intravenously (i.v.), or by orthotopic injection to the colon, under direct vision. The mice thereafter are administered with the tested compounds i.d., i.v., intraperitoneally (i.p.), per os (p.o.) in drinking water, or intrarectally. The size of tumor and the survival of treated mice are compared to a control group (of non-treated mice). Pathological examination is used to reveal possible metastases in mice.

### Example 5

### Ceramide, Sphingomyelin and Glucocerebroside Measurements

Ceramide is a precursor for many sphingolipids in cells, amongst which are sphingomyelin and glucocerebroside. Elevated levels of ceramide in cells lead to apoptosis. Thus, preventing the syntheses of sphingolipids from ceramide will raise the amount of the latter and lead to cell apoptosis.

To measure the concentrations of ceramide, sphingomyelin and glucocerebroside, cancer cells were first incubated with the fluorescent reagent BodiPy-12-Cer 0.5 μM/well. The cells were thereafter incubated with the tested 3-substituted vinylboronate, at elevating concentrations. The cells were thereafter washed and centrifuged, and the upper layer was removed. The residue was extracted with 1:2 CH₂Cl₂:MeOH, the solvents were removed from the organic extract and ethanol was added to the obtained dried samples. The samples were analyzed using HPLC with a spectrofluorometer detector to quantify ceramide, sphingomyelin and glucocerebroside in the cells treated with the tested compound, as compared to non-treated control cells (denoted “E1 cells”). The results are presented in FIGS. 3A-3C.

FIGS. 3A and 3B show a dose-response effect of the tested exemplary 3-hydroxy vinylboronate (E1) on the level of glucocerebroside (GC), with increasing concentrations of E1, in ARH-77 cells, resulting in decreasing level of GC. The results further show increased level of ceramide and decreased level of sphingomyelin (SPM) in ARH-77 cells incubated with 5 and 8 μM of Compound E1. As shown in FIG. 3C, similar effects on GC, SPM and ceramide levels were observed in HT-29 cells incubated with increasing concentrations of the tested compound E1.

These results show that 3-substituted vinylboronates possess significant ability to modulate sphingolipid metabolism: to increase ceramide levels and decrease the levels of sphingomyelin and glucocerebroside in cancer cells, suggesting that the reduced survival of cancer cells shown in Example 4 hereinabove may result from involvement in sphingolipids metabolism.

In addition, incubation of cells within Bodipy-12-CER for 72 hours in the absence of any agent revealed diverse percentiles of SPM formation, as follows: A2780-45%, A2780cisR-60%, HT-29-80%, and CRL-5803-48% (see also FIG. 4). Further studies indicated that normal lymphoblast cells revealed SPM formation that ranges between 5-15%. The obtained results further indicate that HT-29 cells without drug treatment synthesize about twice the amount of sphingomyelin, as compared to other cancer cell lines like A2780 (ovarian carcinoma) or CRL5803 (non-small cell lung carcinoma). The only tested cancer cell line that synthesizes similar amounts of sphingomyelin was the A2780 cisplatin-resistant line (A2780cisR).

### Example 6

### Enzymes Inhibition Study

The following proteases were used in these studies: Trypsin, Elastase, alpha-Chymotrypsin and Leucine aminopeptidase.

alpha-1-Antitrypsin is the commercial inhibitor for the first three enzymes.

Phenyl boronic acid is also an inhibitor (less potent).

The substrates of the enzymes are: BApNA for Trypsin, SucAla₃-PNA for Elastase, BTpNA for alpha-Chymotrypsin, and L-leucine-p-Nitroanilide for Leucine aminopeptidase.

The tested compound was E1 described supra.

Trypsin and elastase inhibition was tested with four concentrations of E1: 10 μM, 1 μM, 0.1 μM and 0.01 μM. alpha-Chymotrypsin and leucine aminopeptidase inhibition was tested with three concentrations of E1: 50 μM, 10 μM and 1 μM.

The experiments were conducted according to the following illustrative scheme:

Buffer+enzyme(37° C., 5 minutes incubation)→add inhibitor(37° C., 10 minutes incubation)→add substrate(37° C., 30 minutes incubation)→add 30% acetic acid→check at λ=410 nm.

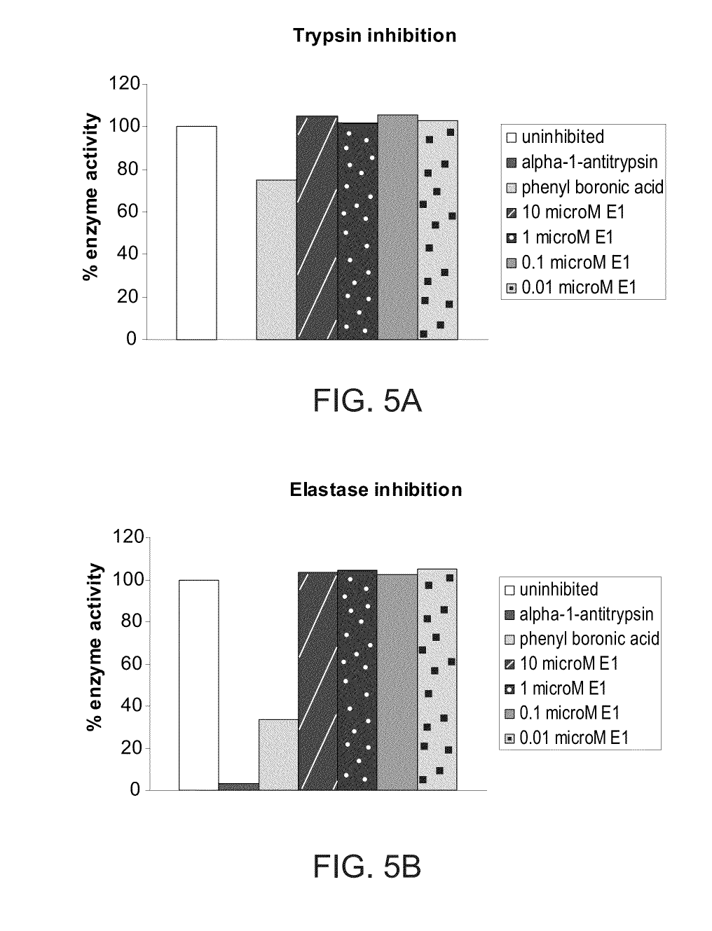

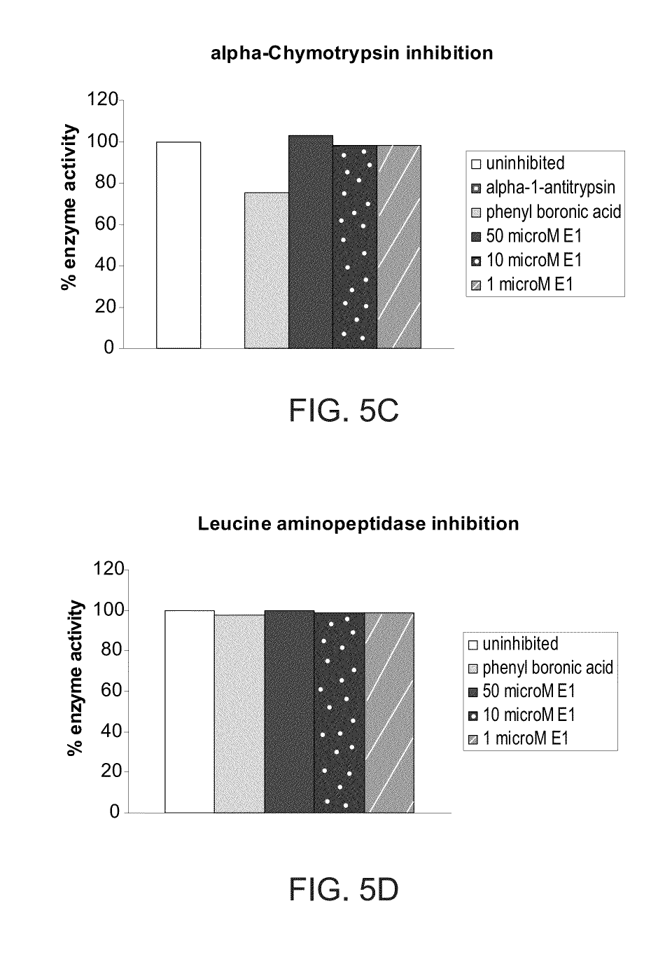

The results are present in FIGS. 5A-D and clearly indicate that Compound E1 does not inhibit the activity of the tested proteases.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that to section headings are used, they should not be construed as necessarily limiting.

1-51. (canceled)

52. A method of treating cancer, the method comprising administering to a subject in need thereof a therapeutically effective amount of a 3-substituted vinylboronate.

53. The method of claim 52, wherein said cancer is selected from the group consisting of colon cancer, rectal cancer and colorectal cancer.

54. The method of claim 52, wherein said cancer is myeloma.

55. The method of claim 52, wherein said 3-substituted vinylboronate is a 3-hydroxy vinylboronate.

56. The method of claim 52, wherein said 3-substituted vinylboronate has the general Formula I: 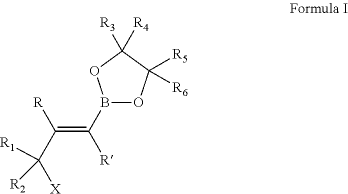
wherein:
X is selected from the group consisting of hydroxy, amine, amide, carboxy, thiocarboxy, thiol, alkoxy, thioalkoxy, aryloxy, thioaryloxy, sulfonamide, thioamide, carbamate, thiocarbamate, sulfonate, heteroalicyclic, heteroaryl, guanidinyl and guanyl;
R is selected from the group consisting of hydrogen, alkyl, alkenyl, cycloalkyl, aryl, heteroaryl, heteroalicyclic, alkoxy, aryloxy, thioalkoxy, thioaryloxy and alkylamino;
R′ is hydrogen;
R₁ and R₂ are each independently selected from the group consisting of hydrogen, alkyl, alkenyl, alkynyl, cycloalkyl, aryl, heteroaryl, and heteroalicyclic, or alternatively, R₁ and R₂ form a 4-, 5- or 6-membered saturated or unsaturated, alicyclic or heteroalicyclic ring, optionally fused to another ring; and
R₃-R₆ are each independently selected from the group consisting of alkyl, cycloalkyl, and aryl, or, alternatively, two of R₃-R₆ form a 4-, 5- or 6-membered saturated or unsaturated, alicyclic or heteroalicyclic ring.

57. The method of claim 56, wherein X is selected from the group consisting of hydroxy, amine, amide and carboxy.

58. The method of claim 56, wherein R is alkyl.

59. The method of claim 58, wherein R is an alkyl being at least 4 carbon atoms in length.

60. The method of claim 56, wherein each of R₃-R₆ is alkyl.

61. The method of claim 56, wherein R₁ and R₂ are each independently selected from the group consisting of alkyl, cycloalkyl and aryl.

62. The method of claim 52, wherein said 3-substituted vinylboronate is selected from the group consisting of Compounds E1, E2, E3, A5, A7, E5, E6 and E7:

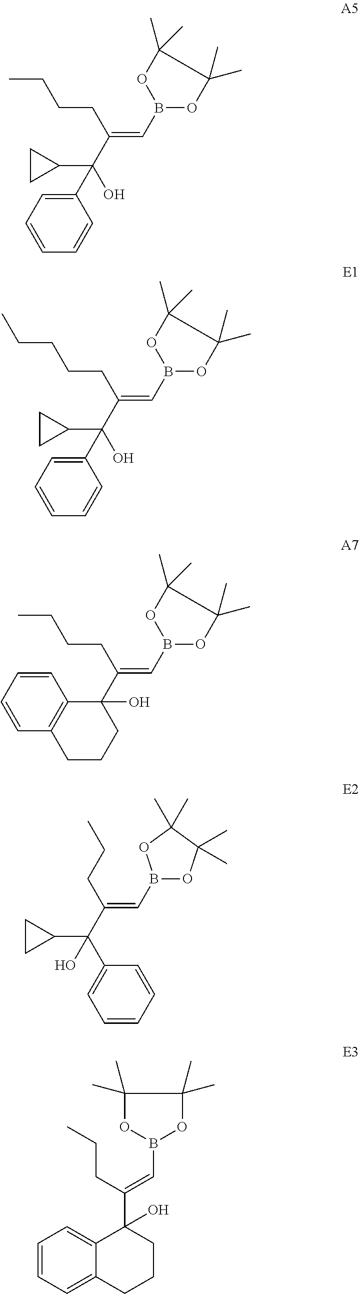

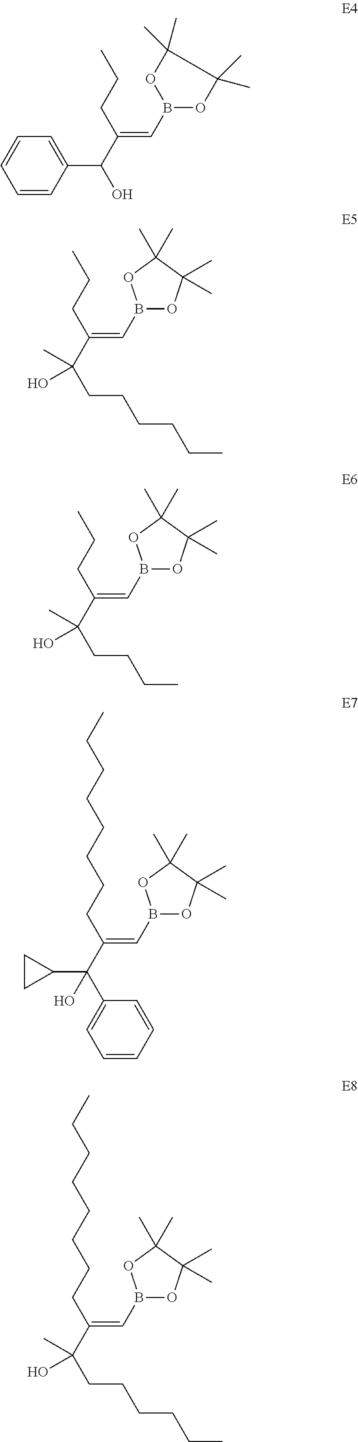

63. A pharmaceutical composition comprising a 3-substituted vinylboronate and a pharmaceutically acceptable carrier.

64. The pharmaceutical composition of claim 63, being packaged in a packaging material and identified in print, in or on said packaging material, for use in the treatment of cancer.

65. The pharmaceutical composition of claim 64, wherein said cancer is selected from the group consisting of colon cancer, rectal cancer and colorectal cancer.

66. The pharmaceutical composition of claim 64, wherein said cancer is myeloma.

67. The pharmaceutical composition of claim 63, wherein said 3-substituted vinylboronate is a 3-hydroxy vinylboronate.

68. The pharmaceutical composition of claim 63, wherein said 3-substituted vinylboronate has the general Formula I: 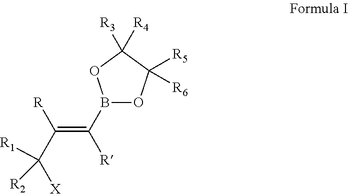
wherein:
X is selected from the group consisting of hydroxy, amine, amide, carboxy, thiocarboxy, thiol, alkoxy, thioalkoxy, aryloxy, thioaryloxy, sulfonamide, thioamide, carbamate, thiocarbamate, sulfonate, heteroalicyclic, heteroaryl, guanidinyl and guanyl;
R is selected from the group consisting of hydrogen, alkyl, alkenyl, cycloalkyl, aryl, heteroaryl, heteroalicyclic, alkoxy, aryloxy, thioalkoxy, thioaryloxy and alkylamino;
R′ is hydrogen;
R₁ and R₂ are each independently selected from the group consisting of hydrogen, alkyl, alkenyl, alkynyl, cycloalkyl, aryl, heteroaryl, and heteroalicyclic, or alternatively, R₁ and R₂ form a 4-, 5- or 6-membered saturated or unsaturated, alicyclic or heteroalicyclic ring, optionally fused to another ring; and
R₃-R₆ are each independently selected from the group consisting of alkyl, cycloalkyl, and aryl, or, alternatively, two of R₃-R₆ form a 4-, 5- or 6-membered saturated or unsaturated, alicyclic or heteroalicyclic ring.

69. The pharmaceutical composition of claim 68, wherein X is selected from the group consisting of hydroxy, amine, amide and carboxy.

70. The pharmaceutical composition of claim 68, wherein R is alkyl.

71. The pharmaceutical composition of claim 70, wherein R is an alkyl being at least 4 carbon atoms in length.

72. The pharmaceutical composition of claim 68, wherein each of R₃-R₆ is alkyl.

73. The pharmaceutical composition of claim 68, wherein R₁ and R₂ are each independently selected from the group consisting of alkyl, cycloalkyl and aryl.

74. The pharmaceutical composition of claim 73, wherein at least one of R₁ and R₂ is an alkyl being at least 4 carbon atoms in length.

75. The pharmaceutical composition of claim 52, wherein said 3-substituted vinylboronate is selected from the group consisting of Compounds E1, E2, E3, A5, A7, E5, E6 and E7:

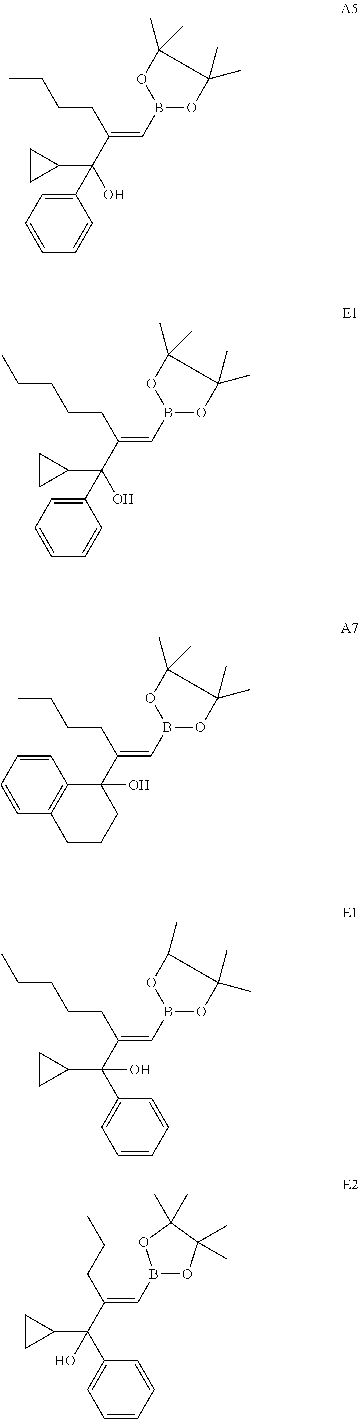

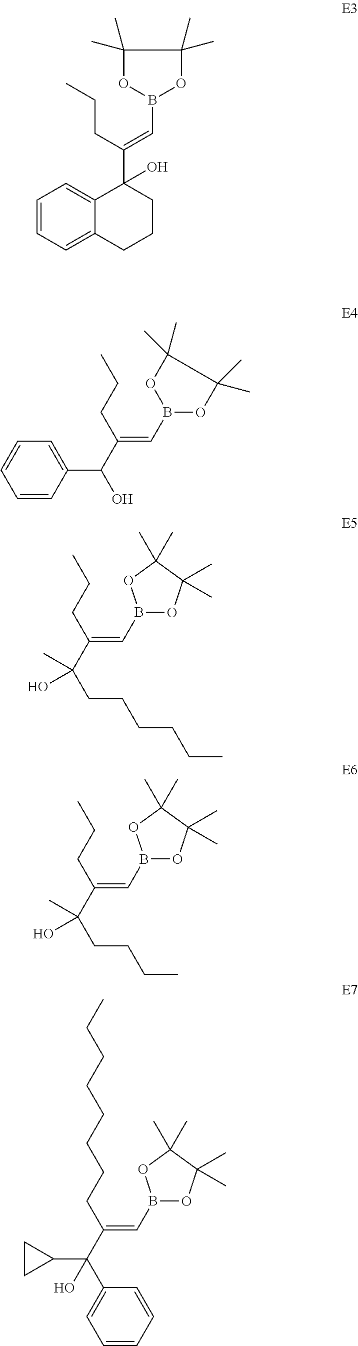

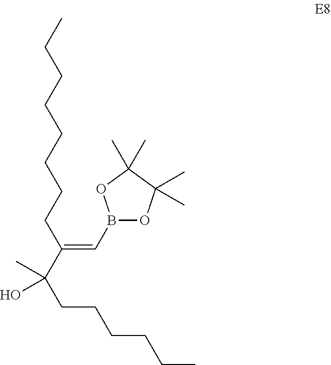 [SMILES: CCCCCCCC/C(=C\B1OC(C)(C)C(C)(C)O1)C(C)(O)CCCCCC]

76. A method of modulating sphingolipid metabolism in cancer cells, the method comprising contacting the cells with an effective amount of a 3-substituted vinylboronate.

77. The method of claim 76, wherein said 3-substituted vinylboronate is a 3-hydroxy vinylboronate.

78. The method of claim 76, wherein said 3-substituted vinylboronate has the general Formula I: 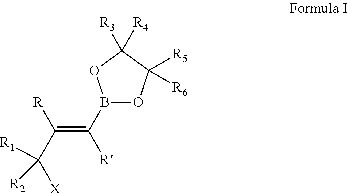
wherein:
X is selected from the group consisting of hydroxy, amine, amide, carboxy, thiocarboxy, thiol, alkoxy, thioalkoxy, aryloxy, thioaryloxy, sulfonamide, thioamide, carbamate, thiocarbamate, sulfonate, heteroalicyclic, heteroaryl, guanidinyl and guanyl;
R is selected from the group consisting of hydrogen, alkyl, alkenyl, cycloalkyl, aryl, heteroaryl, heteroalicyclic, alkoxy, aryloxy, thioalkoxy, thioaryloxy and alkylamino;
R′ is hydrogen;
R₁ and R₂ are each independently selected from the group consisting of hydrogen, alkyl, alkenyl, alkynyl, cycloalkyl, aryl, heteroaryl, and heteroalicyclic, or alternatively, R₁ and R₂ form a 4-, 5- or 6-membered saturated or unsaturated, alicyclic or heteroalicyclic ring, optionally fused to another ring; and
R₃-R₆ are each independently selected from the group consisting of alkyl, cycloalkyl, and aryl, or, alternatively, two of R₃-R₆ form a 4-, 5- or 6-membered saturated or unsaturated, alicyclic or heteroalicyclic ring.

79. The method of claim 78, wherein X is selected from the group consisting of hydroxy, amine, amide and carboxy.

80. The method of claim 78, wherein R is alkyl.

81. The method of claim 80, wherein R is an alkyl being at least 4 carbon atoms in length.

82. The method of claim 78, wherein each of R₃-R₆ is alkyl.

83. The method of claim 78, wherein R₁ and R₂ are each independently selected from the group consisting of alkyl, cycloalkyl and aryl.

84. The method of claim 83, wherein at least one of R₁ and R₂ is an alkyl being at least 4 carbon atoms in length.

85. The method of claim 83, wherein at least one of R₁ and R₂ is a cycloalkyl.

86. The method of claim 76, wherein said 3-substituted vinylboronate is selected from the group consisting of Compounds E1, E2, E3, A5, A7, E5, E6 and E7:

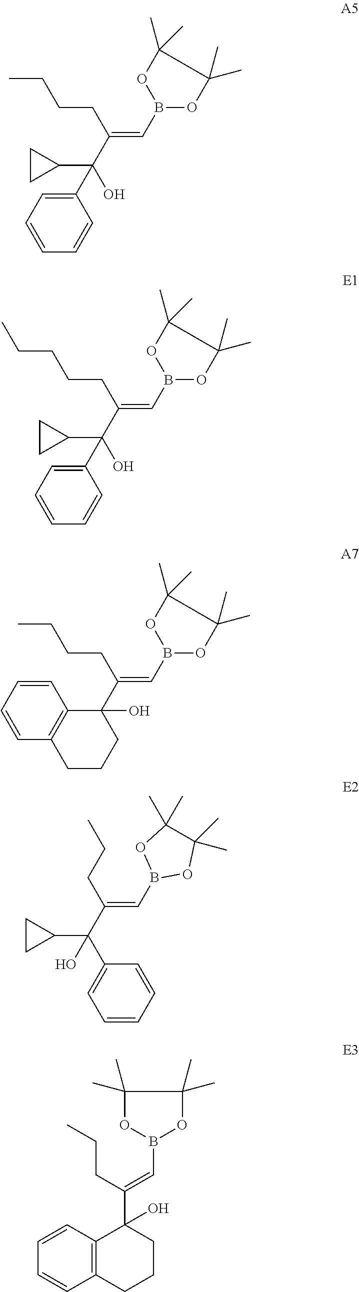

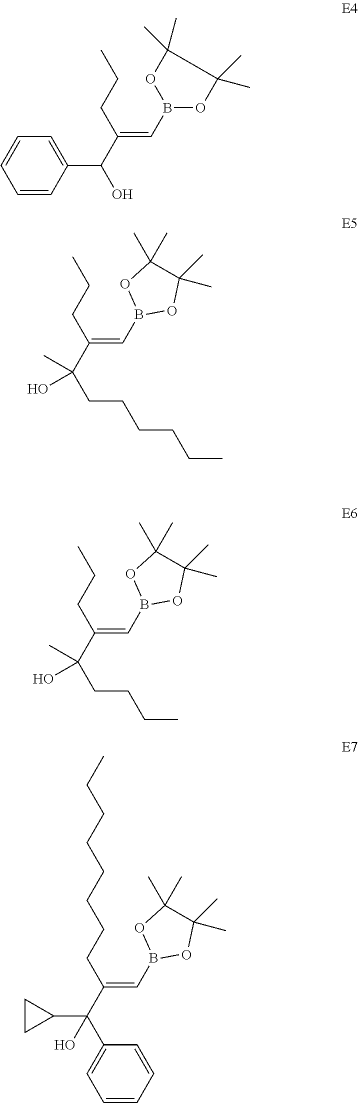

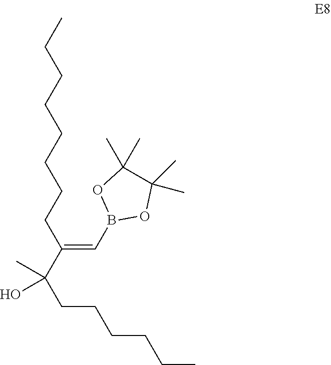 [SMILES: CCCCCCCC/C(=C\B1OC(C)(C)C(C)(C)O1)C(C)(O)CCCCCC]

87. A 3-substituted vinylboronate compound having the general Formula I: 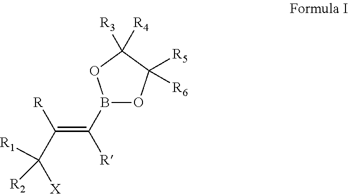
wherein:
X is selected from the group consisting of hydroxy, amine, amide, carboxy, thiocarboxy, thiol, alkoxy, thioalkoxy, aryloxy, thioaryloxy, sulfonamide, thioamide, carbamate, thiocarbamate, sulfonate, heteroalicyclic, heteroaryl, guanidinyl and guanyl;
R is selected from the group consisting of hydrogen, alkyl, alkenyl, cycloalkyl, aryl, heteroaryl, heteroalicyclic, alkoxy, aryloxy, thioalkoxy, thioaryloxy and aminoalkyl;
R′ is hydrogen;
R₁ and R₂ are each independently selected from the group consisting of hydrogen, alkyl, alkenyl, alkynyl, cycloalkyl, aryl, heteroaryl, and heteroalicyclic, or alternatively, R₁ and R₂ form a 4-, 5- or 6-membered saturated or unsaturated, alicyclic or heteroalicyclic ring, optionally fused to another ring; and
R₃-R₆ are each independently selected from the group consisting of alkyl, cycloalkyl, and aryl, or, alternatively, two of R₃-R₆ form a 4-, 5- or 6-membered saturated or unsaturated, alicyclic or heteroalicyclic ring,
provided that at least of R, R₁ and R₂ is an alkyl being at least 6 carbon atoms in length.

88. The compound of claim 87, wherein X is selected from the group consisting of hydroxy, amine, amide and carboxy.

89. The compound of claim 87, wherein X is hydroxy.

90. The compound of claim 87, wherein R is alkyl.

91. The compound of claim 90, wherein R is said alkyl being at least 6 carbon atoms in length.

92. The compound of claim 87, wherein each of R₃-R₆ is alkyl.

93. The compound of claim 87, wherein R₁ and R₂ are each independently selected from the group consisting of alkyl, cycloalkyl and aryl.

94. The compound of claim 93, wherein at least one of R₁ and R₂ is said alkyl being at least 6 carbon atoms in length.

95. The compound of claim 93, wherein at least one of R₁ and R₂ is a cycloalkyl.

96. The compound of claim 87, being selected from the group consisting of Compounds E5, E7 and E8.

97. The compound:

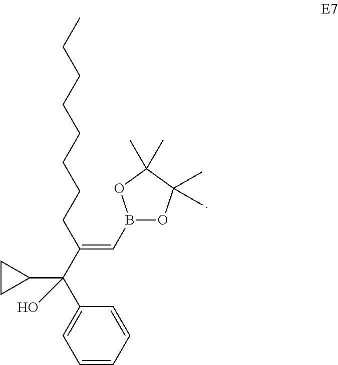 [SMILES: CCCCCCCC/C(=C\B1OC(C)(C)C(C)(C)O1)C(O)(c1ccccc1)C1CC1]

98. A 3-substituted vinylboronate having the general Formula II: 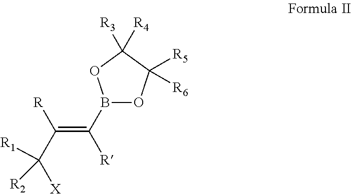
wherein:
X is selected from the group consisting of amine, amide, carboxy, thiocarboxy, thiol, alkoxy, thioalkoxy, aryloxy, thioaryloxy, sulfonamide, thioamide, carbamate, thiocarbamate, sulfonate, heteroalicyclic, heteroaryl, guanidinyl and guanyl;
R is selected from the group consisting of hydrogen, alkyl, alkenyl, cycloalkyl, aryl, heteroaryl, heteroalicyclic, alkoxy, aryloxy, thioalkoxy, thioaryloxy, aminoalkyl and amine;
R′ is hydrogen;
R₁ and R₂ are each independently selected from the group consisting of hydrogen, alkyl, alkenyl, alkynyl, cycloalkyl, aryl, heteroaryl, and heteroalicyclic, or alternatively, R₁ and R₂ form a 4-, 5- or 6-membered saturated or unsaturated, alicyclic or heteroalicyclic ring, optionally fused to another ring; and
R₃-R₆ are each independently selected from the group consisting of alkyl, cycloalkyl, and aryl, or, alternatively, two of R₃-R₆ form a 4-, 5- or 6-membered saturated or unsaturated, alicyclic or heteroalicyclic ring.

99. The compound of claim 98, wherein R is alkyl.

100. The compound of claim 98, wherein each of R₃-R₆ is alkyl.

101. The compound of claim 98, wherein X is selected from the group consisting of amine, amide and carboxy.

102. The compound of claim 98, wherein R₁ and R₂ are each independently selected from the group consisting of alkyl, cycloalkyl and aryl.

103. A pharmaceutical composition comprising the compound of claim 87 and a pharmaceutically acceptable carrier.

104. The composition of claim 103, being packaged in a packaging material and identified in print, in or on said packaging material, for use in the treatment of cancer.
